(19) United States
(12) Patent Application Publication
(10) Pub. No.: US 2017/0204450 A1
(43) Pub. Date: Jul. 20, 2017

(54) METHOD AND ENZYME SOLUTION FOR FLOW-CYTOMETRIC DETECTION OF LIGHT CHAIN RESTRICTION

(71) Applicant: RUPRECHT-KARLS-UNIVERSITAET HEIDELBERG, Heidelberg (DE)

(72) Inventors: Stefan KRIENKE, Eppelheim (DE); Michael HUNDEMER, Edenkoben (DE)

(73) Assignee: Ruprecht-Karls-Universitaet Heidelberg

(21) Appl. No.: 15/312,780

(22) PCT Filed: May 28, 2015
(86) PCT No.: PCT/EP2015/061861

§ 371 Date: Nov. 21, 2016

(30) Foreign Application Priority Data
May 28, 2014 (DE) 20 2014 004 473.2

(51) Int. Cl. C12Q 1/37; G01N 33/50; C12N 9/52; G01N 15/14; C12N 9/76; C12N 9/64

(52) U.S. Cl. CPC C12Q 1/37; C12Y 304/24024; C12Y 304/24021; C12Y 304/21004; C12N 9/6427; C12N 9/6416; C12N 9/52; G01N 15/1404; G01N 33/5047; G01N 2333/96433; G01N 2333/96486

(57) ABSTRACT

The invention relates to an enzyme solution, which is used preferably for pre-treating mammalian cells for flow-cytometric detection of light chain restriction of the cells, the enzyme solution comprising at least two proteases, which are proteolytic and also collagenolytic, in a buffer. Preferably, the enzyme solution comprises trypsin, collagenase 4, dispase and astacin. The invention also relates to uses of the enzyme solution and methods for flow-cytometric detection of light chain restriction of cells.

The invention relates to a method for detecting a light chain restriction of plasma and B cells from bone marrow (preferably, but not exclusively) in peripheral blood, bone marrow or liquor with the aid of a special enzyme solution for flow-cytometric analysis.

Flow cytometry (fluorescence activated cell sorting=FACS) is a method of cell analysis, which is used for automated investigation of peripheral blood or bone marrow cells. The devices used for this investigation, are called flow cytometers.

In flow cytometry, the cells to be investigated flow through a thin measuring chamber (flow cell) in a special buffer. The reflection of a laser beam directed onto these cells generates scattered light (light scatter) that is characteristic for each cell type. The more voluminous a cell and the more differentiated the structures in the cytoplasm, the greater is the scattered light produced. By measuring the scattered light, it is therefore possible to make a broad statement about the number and distribution of the various cell types in the sample in a relatively simple manner.

Thus, granulocytes (a certain cell type of white blood cells), which have a rough surface and many vesicles in the interior thereof, scatter considerably more light than the very smooth B or T cells (B or T lymphocytes). The forward scattered light (FSC=Forward Scatter) is a measure for the diffraction of the light in a shallow angle and depends on the volume of the cell. The sidewards scattered light (SSC=Sidewards Scatter) is a measure for the diffraction of the light at right angles, which is influenced by the granularity of the cell, the size and structure of the cell nucleus thereof and the quantity of vesicles in a cell. Using these two parameters, the cells of the blood can be differentiated very well, even unstained, for example.

More accurate characterisations of the individual cell types is made by means of antibodies, which bind specifically to cell surfaces and are coupled to a fluorescent dye (fluorochrome) for example. For this purpose, the prepared cells are incubated with appropriate antibodies and subsequently the fluorescences are analysed in the flow cytometer.

The antibodies are for the most part directed against certain surface proteins of CD classification (CD=Cluster of Differentiation). By using differently coloured lasers and above all, corresponding filters, it is possible to detect various markers simultaneously in a measurement, provided that the wavelengths of the emitted fluorescent light of the used fluorophores differ (multiple dyeing).

Flow cytometry has become a valuable tool for diagnosis and characterisation of tumour diseases, particularly haematological neoplasias. Establishing the diagnosis and statements on prognosis and progression of malignant lymphatic diseases and typing acute leukaemia are among the most important fields of use of this method. In addition to investigating blood, bone marrow and liquor, this method also includes the characterisation of cells from biopsy material.

Plasma cells are special white blood cells of the immune system and are used for producing and secreting immunoglobulins (antibodies). They correspond to the last stage of differentiation of the B cell line and appear in the light microscope as large oval cells with eccentrically located cell nucleus. Immunoglobulins (apart from IgM) have a common basic structure, consisting of two heavy (H) and two light (L) chains. The chains are bonded to one another by disulphide bridges. The heavy and light chains are amino terminal at the same ends. In the case of L chains, a distinction is made between the kappa and the lambda light chain types. Each immunoglobulin molecule has either two kappa or two lambda L chains, as B lymphocytes can only form one L chain type. These light chains are also found on the surface and the cytoplasm of plasma cells and B lymphocytes.

Multiple myeloma is a malignant disease (tumour) of plasma cells, characterised by a slow reproduction of plasma cells. All malignant plasma cells produce the same (=monoclonal) antibodies, an important feature of this disease.

B-cell non-Hodgkin lymphoma is a malignant disease of B lymphocytes, which is expressed for the most part in lymphadenopathy. In some cases, such as e.g. in the case of chronically lymphatic leukaemia, these malignant cells can also be detected in blood or bone marrow, however.

In the case of a suspicion or the presence of malignant plasma cells/B lymphocytes in peripheral blood, liquor and bone marrow, flow cytometry can verify this. To do this, it is necessary to carry out the detection of a light chain restriction on the surface or in the cytoplasm of plasma cells/B lymphocytes, in order to differentiate between polyclonal (reactive) and monoclonal cell populations. As plasma cells/B lymphocytes express either K or A light chains, malignant cells only show the expression of one of the two light chains, as they are of clonal origin.

Up to the present, a clear distinction between malignant and physiological plasma cells and to some extent also B lymphocytes in flow cytometric analysis was only possible by means of the different expression of aberrant surface antigens, as the analysis of the clonal light chains was not possible for technical reasons.

In current investigations, it was possible to show that an aberrant expression of plasma cell surface antigens not only occurs on malignant, but rather also on physiological plasma cells and as a result, this method is only to be used in a limited manner with regards to the statement of whether malignant or physiological plasma cells are present in the case of the respective patient.

By means of the enzymatic preparation according to the invention of bone marrow samples by means of proteases (such as collagenases and peptidases), it is possible for patients with plasma and B cell diseases, to detect the light chain restriction of the underlying malignant cells by means of flow cytometric analysis. Eventually, the epitope (the binding site) of the light chains on the plasma cells, which is recognised by antibodies against light chains in flow cytometric analysis, is masked owing to the enormously high protein concentration in the bone marrow of patients with plasma B cell diseases, which concentration comes about due to the immunoglobulin-secreting malignant cells.

Probably, lytic biochemical processes are triggered by the enzymes, which lead to the exposure of the relevant light chain epitopes on the plasma B cells. It is possible by means of the enzyme solution according to the invention, on the one hand to make a reliable distinction between malignant and physiological cells and on the other hand to increase the sensitivity of the flow cytometric analysis for detecting small malignant clones, particularly in the case of prognostic and therapy-relevant minimal residual disease.

Enzyme solutions with peptidases for FACS analyses are known to some extent (inter alia WO1994025487 A1, DE102007008650 B4), but these are used in terms of the composition and use thereof for eluting or dissipating from a cell cluster or from in-vitro cultures exclusively, for example for conversion to a single-cell suspension. This buffer for preparation for FACS analyses cannot expose any masked surface molecules for antibody marking.

It is the object of the present invention to overcome the disadvantages of existing analysis buffers and to provide a simple FACS analysis buffer with enzyme solution for detecting light chain restriction.

The object of this invention is achieved by the enzyme solutions, uses and methods in the claims. Advantageous embodiments are to be found in the description.

Subject of the invention is an enzyme solution, which is used preferably for pre-treating mammalian cells for flow-cytometric detection of light chain restriction of the cells, the enzyme solution comprising at least two proteases, which are proteolytic and also collagenolytic, in a buffer.

Preferably, the enzyme solution comprises at least three enzymes selected from trypsin, collagenase 4, dispase and astacin. Preferably, the enzyme solution comprises trypsin, collagenase 4, dispase and astacin. As used herein, the term “astacin” refers to an enzyme from the astacin family of metalloproteases. Thus, the enzyme solution “comprising astacin” should comprise at least one enzyme from the astacin family of metalloproteases.

Antigens, in this case specific light chains of the lymphocytes, can be exposed and coloured with the inventive enzyme solution. This was only limitedly possible until now, even though it is highly important as the proof for malignancy can only be provided with evidence of light chain restriction.

Preferably, the cells are from a cell sample selected from the group comprising blood plasma, bone marrow, lymph, liquor, endolymph, vitreous humour, synovial fluid, pleural fluid, pericardial fluid (liquor pericardii), peritoneal fluid or a combination thereof.

Preferably, the buffer is phosphate buffered salt (PBS). Preferably, the PBS buffer does not comprise calcium and/or magnesium. Preferably, the enzyme solution comprises a cell culture medium, such as RPMI1640. The cell culture medium may comprise fetal calf serum (FCS). The enzyme solution may also comprise other common components for pre-treating cells, such as EDTA.

Preferably, the proteases comprise a mixture of preferably trypsin, collagenase 4, dispase and astacin in a buffer, preferably phosphate buffered salt solution (PBS).

Preferably, the enzyme solution is present in the sample tube before or after the cell sample that can be introduced.

Preferably, trypsin is present in a concentration of 0.25-25 mg/ml, preferably 1.25-2.5 mg/ml, and/or

collagenase 4 is present in a concentration of 0.05-2 mg/ml, preferably 0.05-0.2 mg/ml, and/or

dispase is present in a concentration of 0.05-2 mg/ml, preferably 0.05-0.2 mg/ml, and/or

astacin is present in a concentration of 0.05-25 mg/ml, preferably 0.05-0.2 mg/ml. Preferably, astacin is present in a concentration of 0.3-times 0.5-times.

Preferably, the enzyme solution comprises

0.25-25 mg/ml, preferably 1.25-2.5 mg/ml trypsin,

0.05-2 mg/ml, preferably 0.05-0.2 mg/ml collagenase 4,

0.05-2 mg/ml, preferably 0.05-0.2 mg/ml dispase and

0.05-25 mg/ml, preferably 0.05-0.2 mg/ml astacin.

In a specific embodiment, the enzyme solution does not comprise other proteases or other enzymes.

Another subject of the invention is the use of the enzyme solution for pre-treating cells in a flow-cytometric detection method. Preferably, the flow-cytometric detection method is for determining a light chain restriction of plasma cells and/or B lymphocytes. Preferably, the plasma cells and/or B lymphocytes are from bone marrow, liquor or peripheral blood.

Preferably, the method is for detecting malignant cells. Preferably, the method is a diagnostic method, preferably for diagnosing multiple myeloma or B-cell non-Hodgkin lymphoma.

Another subject of the invention is an in vitro method for determining a light chain restriction of plasma cells and/or B lymphocytes by a flow-cytometry detection method, comprising:
(a) providing plasma cells and/or B lymphocytes, which preferably have been obtained from the mammalian body, preferably from bone marrow, liquor or peripheral blood,
(b) contacting the plasma cells and/or B lymphocytes with the enzyme solution,
(c) subjecting the cells to the flow-cytometric detection method, and
(d)determining a light chain restriction of the cells.

In step (a), the cells are preferably isolated from the mammalian body, such that a cell pellet is obtained. In step (b), the cells are incubated with the enzyme solution for a sufficient time such that the proteases can act as desired on the cell. For example, the cells are incubated with the enzyme solution for 15 to 90 min, preferably at a temperature between 15 and 60° C., preferably between 20 and 40° C. Typically, step (c) comprises staining of the cells with monoclonal antibodies.

Another subject of the invention is a method for flow-cytometric detection of light chain restriction comprising the steps of:

remove peripheral blood or bone marrow from an EDTA tube and transfer to a falcon tube, erythrocytolysis by means of distilled water and balancing by means of ten-times-concentrated phosphate buffer solution (PBS), centrifuge of the cells, absorb the cells in PBS, transfer to Eppendorf vessels and pellet again, resuspend the pelleted cells in the inventive enzyme solution, stop the enzyme reaction after the incubation time by a rinsing step with PBS, absorb the cells in PBS and staining of the blood/bone marrow aspirate with the monoclonal antibodies for flow cytometric analysis.

The preparation of the cells to be investigated is made in a manner, which is obvious for the person skilled in the art and known from the prior art, and which is illustrated here by way of example:

Up to 2 ml of peripheral blood or bone marrow are removed from an EDTA tube (ethylenediaminetetraacetic acid preincubated tube for the anticoagulation of whole blood) and transferred to a 50 ml falcon tube. Without or after double hypotonic erythrocytolysis by means of distilled water for 25 seconds in each case and corresponding balancing by means of ten-times-concentrated phosphate buffer solution (PBS), the cells are centrifuged down (pelleted). Subsequently, these cells are absorbed in 1.2 to 1.6 ml PBS, transferred to 400 μl portions in 1.5 ml Eppendorf vessels in each case and pelleted again. These cell pellets are resuspended in 300 μl total volume in the enzyme solution according to the invention. After the respective incubation time (15 to 90 min at room temperature or 37° C.), the enzyme reaction is stopped by a rinsing step with PBS. The cells are initially absorbed in 100 μl PBS and subsequently the staining of the blood/bone marrow aspirate with the monoclonal antibodies for flow cytometric analysis is undertaken.

The enzyme solution according to the invention consists by way of example of:

Trypsin (0.25-25 mg/ml PBS) without calcium and magnesium (w/o Ca&Mg), collagenase 4 (0.05-2 mg/ml) cell culture medium, such as for example RPMI1640 incl. 10% fetal calf serum (FCS), dispase (0.05-2 mg/ml) RPMI incl. 10% FCS and astacin (such as for example accutase 0.3-times-5-times in PBS w/o Ca&Mg+0.5 mM EDTA)

What is claimed is:

1. An enzyme solution, for pre-treating mammalian cells for flow-cytometric detection of light chain restriction of the cells, the enzyme solution comprising at least two proteases, which are proteolytic and also collagenolytic, in a buffer.

2. The enzyme solution of claim 1 comprising at least three enzymes selected from trypsin, collagenase 4, dispase and astacin.

3. The enzyme solution of claim 2 comprising trypsin, collagenase 4, dispase and astacin.

4. The enzyme solution according to claim 1, wherein the cells are from a cell sample selected from the group consisting of blood plasma, bone marrow, lymph, liquor, endolymph, vitreous humour, synovial fluid, pleural fluid, pericardial fluid (liquor pericardii), peritoneal fluid and a combination thereof.

5. The enzyme solution according to claim 1, wherein the buffer is phosphate buffered salt (PBS).

6. The enzyme solution according to claim 1, wherein trypsin is present in a concentration of 0.25-25 mg/ml, preferably 1.25-2.5 mg/ml.

7. The enzyme solution according to claim 1, wherein collagenase 4 is present in a concentration of 0.05-2 mg/ml, preferably 0.05-0.2 mg/ml.

8. The enzyme solution according to claim 1, wherein dispase is present in a concentration of 0.05-2 mg/ml, preferably 0.05-0.2 mg/ml.

9. The enzyme solution according to claim 1, wherein astacin is present in a concentration of 0.05-25 mg/ml, preferably 0.05-0.2 mg/ml.

10. The enzyme solution according to claim 1, comprising:
0.25-25 mg/ml trypsin,
0.05-2 mg/ml collagenase 4,
0.05-2 mg/ml dispase and
0.05-25 mg/ml astacin.

11. A flow-cytometric detection method for pre-treating cells comprising the enzyme solution of claim 1.

12. The method of claim 11, wherein the flow-cytometric detection method determines a light chain restriction of plasma cells and/or B lymphocytes.

13. The method of claim 12, wherein the plasma cells and/or B lymphocytes are from bone marrow, liquor or peripheral blood.

14. An in vitro method for determining a light chain restriction of plasma cells and/or B lymphocytes by a flow-cytometry detection method, comprising:
(a) providing plasma cells and/or B lymphocytes obtained from bone marrow, liquor or peripheral blood,
(b) contacting the plasma cells and/or B lymphocytes with an enzyme solution of claim 1,
(c) subjecting the cells to the flow-cytometric detection method, and
(d) determining a light chain restriction of the cells.

15. A method for flow-cytometric detection of light chain restriction comprising the steps of:
a. removing peripheral blood or bone marrow from an EDTA tube and transferring to a falcon tube,
b. erythrocytolysis by means of distilled water and balancing by means of ten-times-concentrated phosphate buffer solution (PBS), and centrifuging to pellet cells,
c. absorbing the cells in PBS, transferring the absorbed cells to Eppendorf vessels and pelleting the cells again,
d. resuspending the pelleted cells in the enzyme solution according to claim 1,
e. stopping the enzyme reaction after an incubation time by rinsing with PBS, absorbing the cells in PBS and staining the blood/bone marrow aspirate with monoclonal antibodies for flow cytometric analysis.

16. The enzyme solution according to claim 1, wherein trypsin is present in a concentration of 1.25-2.5 mg/ml.

17. The enzyme solution according to claim 1, wherein collagenase 4 is present in a concentration of 0.05-0.2 mg/ml.

18. The enzyme solution according to claim 1, wherein dispase is present in a concentration of 0.05-0.2 mg/ml.

19. The enzyme solution according to claim 1, wherein astacin is present in a concentration of 0.05-0.2 mg/ml.

20. The enzyme solution according to claim 1, comprising:
1.25-2.5 mg/ml trypsin,
0.05-0.2 mg/ml collagenase 4,
0.05-0.2 mg/ml dispase and
0.05-0.2 mg/ml astacin.
